(19) United States
(12) Patent Application Publication
(10) Pub. No.: US 2021/0037872 A1
(43) Pub. Date: Feb. 11, 2021

(54) LIQUID COMPOSITION FOR ORAL USE CONTAINING COLLAGEN PEPTIDE, AND METHOD FOR ALLEVIATING THE ACIDITY OF LIQUID COMPOSITION FOR ORAL USE

(71) Applicant: SUNTORY HOLDINGS LIMITED, Osaka-shi, Osaka (JP)

(72) Inventors: Megumi Okada, Soraku-gun (JP); Takako Imao, Soraku-gun (JP)

(73) Assignee: SUNTORY HOLDINGS LIMITED, Osaka-shi, Osaka (JP)

(21) Appl. No.: 16/967,831

(22) PCT Filed: Jan. 30, 2019
(86) PCT No.: PCT/JP2019/003163

§ 371 Date: Aug. 6, 2020

(30) Foreign Application Priority Data
Feb. 9, 2018 (JP) 2018-022436

(51) Int. Cl. A23L 33/18; C07K 14/78; A23L 2/60; A23L 29/269; A23L 33/125; A23L 2/68

(52) U.S. Cl. CPC A23L 33/18; C07K 14/78; A23L 2/60; A23V 2002/00; A23L 33/125; A23L 2/68; A23L 29/271

(57) ABSTRACT

The present invention provides a liquid oral composition containing collagen peptides in which the acidity is moderated to facilitate oral intake in the pH range of 3.0 to 4.0. The present invention relates to a liquid oral composition containing collagen peptides, welan gum, and an acidulant, the liquid oral composition having a pH of 3.0 to 4.0.

### TECHNICAL FIELD

The present invention relates to a liquid oral composition containing collagen peptides. The present invention also relates to a method of moderating (reducing) the acidity of a liquid oral composition containing collagen peptides.

### BACKGROUND ART

Collagen peptides have been revealed to have various functions such as making the skin beautiful (e.g., improving moisture-retaining properties and elasticity of the skin) and improving blood fluidity, and are nowadays contained in many beverages, foods, cosmetics, and the like.

Oral compositions containing collagen peptides have been provided in the form of various beverages. In order to inhibit growth of microorganisms that cause spoilage during storage, it is important to adjust the pH of beverages to a low value. However, beverages taste acidic in a low pH range, and beverages that are too acidic have an impaired flavor and are thus not suitable for drinking. Studies have been made on methods of reducing the acidity of food. Patent Literature 1 states that D-mannose reduces the strong acidity of cranberry fruit.

### CITATION LIST

### Patent Literature

Patent Literature 1: JP 2012-170342 A

### SUMMARY OF INVENTION

### Technical Problem

Collagen peptides are known to have a pH buffering capacity. Thus, when collagen peptides are added to a liquid oral composition, the impact of the pH buffering capacity cannot be ignored. In particular, a large amount of acidulant is required in order to adjust the pH of a liquid oral composition containing collagen peptides to an antiseptically effective low value (e.g., pH 4.0 or lower). As a result, the liquid oral composition tastes very acidic and thus has a flavor unsuitable for oral intake.

The present invention aims to provide a liquid oral composition containing collagen peptides in which the acidity is moderated to facilitate oral intake in the pH range of 3.0 to 4.0.

### Solution to Problem

As a result of extensive studies to solve the above problem, the present inventors found that adding welan gum to a liquid oral composition containing collagen peptides can effectively moderate the acidity of the composition even when the pH is 3.0 to 4.0.

Specifically, the present invention relates to the following liquid oral composition and the like.

(1) A liquid oral composition containing: a collagen peptide; welan gum; and an acidulant, the liquid oral composition having a pH of 3.0 to 4.0.

(2) The liquid oral composition according to (1) above, wherein the amount of the collagen peptide is 600 to 20000 mg/100 mL.

(3) The liquid oral composition according to (1) or (2) above, wherein the weight ratio of the collagen peptide to the welan gum (collagen peptide/welan gum) is 20 to 100.

(4) The liquid oral composition according to any one of (1) to (3) above, wherein the viscosity is 30 to 500 mPa·s.

(5) The liquid oral composition according to any one of (1) to (4) above, wherein the amount of the welan gum is 50 to 400 mg/100 mL.

(6) The liquid oral composition according to any one of (1) to (5) above, wherein the acidulant includes citric acid or its salt and/or phosphoric acid or its salt.

(7) The liquid oral composition according to any one of (1) to (6) above, further containing a sweetener.

(8) The liquid oral composition according to any one of (1) to (7) above, further containing a proteoglycan and/or elastin peptide.

(9) The liquid oral composition according to any one of (1) to (8) above, which is a beverage.

(10) A method of moderating the acidity of a liquid oral composition, the method including: adding welan gum to a liquid oral composition that contains a collagen peptide and an acidulant and has a pH of 3.0 to 4.0.

### Advantageous Effects of Invention

The present invention can provide a liquid oral composition containing collagen peptides in which the acidity is moderated to facilitate oral intake in the pH range of 3.0 to 4.0.

### DESCRIPTION OF EMBODIMENTS

### <Liquid Oral Composition>

The liquid oral composition of the present invention contains collagen peptides, welan gum, and an acidulant, and has a pH of 3.0 to 4.0.

### <Collagen Peptides>

The collagen peptides used in the present invention can be obtained by hydrolysis of collagen or modified collagen such as gelatin with an enzyme, acid, alkali, or the like. The source and production method of the collagen peptides are not limited. Artificially synthesized collagen peptides can also be used. One type of collagen peptides may be used alone or two or more types of collagen peptides may be used in combination.

Collagen or gelatin as a raw material of the collagen peptides may be one from bovine, swine, chicken, fish, or the like. One or more of these can be used as raw materials. In one embodiment, collagen from fish is preferred. Fish may be saltwater fish or freshwater fish. Examples include tuna (yellowfin), shark, cod, olive flounder, righteye flounder, sea bream, tilapia, salmon, and catfish.

Any enzyme may be used to prepare the collagen peptides as long as the enzyme can cleave peptide bonds of collagen or gelatin. Examples include collagenase, papain, bromelain, actinidine, ficin, cathepsin, pepsin, chymosin, trypsin, and enzymatic preparations in which these enzymes are mixed. The acid may be, for example, hydrochloric acid, sulfuric acid, nitric acid, or the like. The alkali may be, for example, sodium hydroxide, calcium hydroxide, or the like.

In the present invention, an aqueous solution of hydrolyzed collagen peptides may be used as is or a hydrolyzed collagen peptide powder obtained by drying or the like may be used. Alternatively, the aqueous solution subjected to a usual purification treatment may be used in the form of an aqueous solution, a powder, or the like. Commercially available collagen peptides can also be used.

The average molecular weight of the collagen peptides used in the present invention is not limited, but it is preferably 5000 or less, more preferably 4000 or less, still more preferably 3000 or less, yet still more preferably 2000 or less, particularly preferably 1000 or less, most preferably 800 or less. An average molecular weight of 5000 or less is preferred because collagen peptides having such an average molecular weight are highly absorbable in the body when orally taken. While collagen peptides having a smaller average molecular weight are more absorbable in the body, such collagen peptides tend to have a higher buffering capacity than collagen peptides having a larger average molecular weight. Thus, when collagen peptides have a small average molecular weight, it tends to require a larger amount of acidulant in order to adjust the pH of the liquid oral composition to 3.0 to 4.0. In order to effectively moderate the acidity of the liquid oral composition when the composition has a pH of 3.0 to 4.0, the average molecular weight of the collagen peptides is preferably 300 or more, more preferably 350 or more, still more preferably 400 or more. Herein, the range may be any combination of any upper limit and any lower limit.

In one embodiment, the average molecular weight of the collagen peptides is preferably 300 to 5000, more preferably 300 to 4000, still more preferably 300 to 3000, yet still more preferably 350 to 2000, particularly preferably 400 to 1000, most preferably 400 to 800. In the present invention, adding welan gum can effectively moderate the acidity of the liquid oral composition containing collagen peptides having an average molecular weight in the above range even when the composition has a pH of 3.0 to 4.0, thus facilitating oral intake.

Herein, the average molecular weight of the collagen peptides is the weight average molecular weight. Herein, the average molecular weight of the collagen peptides means the value measured by a relative molecular mass measurement method in Chinese National Standards (GB standards) GB/T 22729-2008: oligopeptides powder of marine fish. Yet, substitution products are used as reagents for M, 451 and M, 189.

In this method, substances whose molecular weights are known such as cellular pigment C (cytochrome, M, 6500), Trasylol (aprotinin, M, 12500), Bacillus (bacitracin, M, 1450), glycine-glycine-tyrosine-arginine (M, 451), and glycine-glycine-glycine (M, 189) are measured in advance under the same conditions to obtain a relative molecular mass calibration curve showing a relationship between retention time and logarithm of relative molecular weight. The average molecular weight of the collagen peptides is calculated based on the calibration curve. The average molecular weight herein means the weight average molecular weight calculated in terms of each standard substance according to this method.

Commercially available collagen peptides may be used. Collagen peptides having a preferred average molecular weight can be used. Examples of such commercial products include “Ixos HDL-30DR” (Nitta Gelatin Inc.), “Collapep PU” (Nitta Gelatin Inc.), “TYPE-S” (Nitta Gelatin Inc.), and “HACP” (Jellice Co., Ltd.).

In one embodiment, it is preferred that the collagen peptides in the present invention contain a large amount of Pro-Hyp (prolyl-hydroxyproline (hereinafter “PO”)) and/or Hyp-Gly (hydroxyprolyl-glycine (hereinafter “OG”)) which are dipeptides. It is more preferred that the collagen peptides contain a large amount of PO and OG. Collagen peptides containing such dipeptides are highly useful.

The total amount of PO and OG in the collagen peptides is preferably 0.05 to 10 wt %, more preferably 0.5 to 5.0 wt %.

The amounts of PO and OG can be measured by a known method, for example, using a device such as LC-MS/MS. The collagen peptides in which the total amount of PO and OG is in the above range have a high buffering capacity, and the acidity of a liquid oral composition containing such collagen peptides tends to be particularly strong when the composition has a pH of 3.0 to 4.0. The present invention can effectively moderate the acidity of a liquid oral composition containing such collagen peptides and having a pH of 3.0 to 4.0.

In the liquid oral composition of the present invention, preferably, the amount of the collagen peptides is 600 to 20000 mg/100 mL. When the amount of the collagen peptides is in the above range, the above-described effect of the present invention can be more sufficiently exerted. In addition, when the amount of the collagen peptides is more than 20000 mg/100 mL, a larger amount of welan gum may be added in order to moderate the acidity in the pH range of 3.0 to 4.0. As a result, the liquid oral composition may have a high viscosity, and may not be easily drinkable when provided as a beverage. The amount of the collagen peptides in the liquid oral composition is more preferably 1000 mg/100 mL or more, still more preferably 2000 mg/100 mL or more, while it is more preferably 10000 mg/100 mL or less, still more preferably 7500 mg/100 mL or less. In one embodiment, the amount of the collagen peptides in the liquid oral composition is more preferably 1000 to 10000 mg/100 mL, still more preferably 2000 to 7500 mg/100 mL. The amount means the total amount when several types of collagen peptides are used.

### <Welan Gum>

The welan gum used in the present invention contains polysaccharides obtained from a culture medium of bacteria of the genus Sphingomonas sp. as the main component. Commercially available welan gum can be used. Example of the commercially available welan gum include VISTOP® W available from San-Ei Gen F.F.I., Inc.

The present invention can moderate the acidity of a liquid oral composition containing collagen peptides in the pH range of 3.0 to 4.0 by adding welan gum to the composition. As shown in Examples described later, adding welan gum to a liquid oral composition containing collagen peptides and having a pH of 3.0 to 4.0 moderated (reduced) the acidic mouthfeel of the liquid oral composition (acidity felt in the mouth or acidity of the initial taste) and the acidic aftertaste (acidity of the aftertaste or persisting taste).

In the liquid oral composition of the present invention, preferably, the amount of the welan gum is 50 to 400 mg/100 mL. When the amount of the welan gum is in the above range, the acidity of the liquid oral composition can be further moderated. When the amount of the welan gum is 400 mg/100 mL or less, usually, the liquid oral composition has a drinkable viscosity. In order to moderate the acidity, the amount of the welan gum in the liquid oral composition is preferably 50 mg/100 mL or more, more preferably 80 mg/100 mL or more, still more preferably 100 mg/100 mL or more. In order to moderate the acidity (particularly, acidic aftertaste) and make the viscosity of the liquid oral composition more suitable for drinking, the amount of the welan gum in the liquid oral composition is more preferably 375 mg/100 mL or less, still more preferably 250 mg/100 mL or less, yet still more preferably 200 mg/100 mL or less, particularly preferably 170 mg/100 mL or less. In one embodiment, in order to moderate the acidity and achieve a viscosity suitable for drinking, the amount of the welan gum in the liquid oral composition is more preferably 80 to 375 mg/100 mL, still more preferably 80 to 250 mg/100 mL, yet still more preferably 100 to 200 mg/100 mL, particularly preferably 100 to 170 mg/100 mL.

In the liquid oral composition of the present invention, preferably, the weight ratio of the collagen peptides to the welan gum (collagen peptides/welan gum) is 20 to 100. When the weight ratio of the collagen peptides to the welan gum in the liquid oral composition is in the above range, the acidity of the liquid oral composition can be further moderated. The weight ratio of the collagen peptides to the welan gum is preferably 20 or more, more preferably 25 or more, still more preferably 30 or more, while it is preferably 100 or less, more preferably 90 or less, still more preferably 70 or less, particularly preferably 50 or less. In one embodiment, the weight ratio of the collagen peptides to the welan gum is more preferably 25 to 100, still more preferably 30 to 90, yet still more preferably 30 to 70, particularly preferably 30 to 50.

### <Acidulant>

The liquid oral composition of the present invention contains an acidulant. The acidulant is preferably an acid or its salt usable for food and beverages. For example, the acidulant may be at least one acid or its salt selected from the group consisting of citric acid, phosphoric acid, lactic acid, malic acid, tartaric acid, succinic acid, and fumaric acid, and their salts. The salt is not limited. Examples include a sodium salt, a potassium salt, and a calcium salt. One acidulant may be used alone or two or more acidulants may be used in combination. Only acids in the free acid form or only their salts may be used as acidulants, or free acids and salts may be used in combination.

In one embodiment, the acidulant preferably includes citric acid or its salt, more preferably citric acid. Citric acid or its salt can add natural acidity to the liquid oral composition. At the same time, when citric acid or its salt is used in an attempt to adjust the pH of collagen peptides having a high buffering capacity to 3.0 to 4.0, such an attempt requires a large amount of citric acid or its salt, which may result in a very strong acidity. Use of phosphoric acid or its salt is also preferred because it can effectively reduce the pH while suppressing the acidity. When phosphoric acid or its salt is used, it is preferably used in combination with another acidulant such as citric acid or its salt because such a combination use can prevent addition of astringency or harsh taste to the liquid oral composition.

In the present invention, the acidulant preferably includes citric acid or its salt and/or phosphoric acid or its salt, more preferably citric acid or its salt and phosphoric acid or its salt, or citric acid or its salt. The acidulant still more preferably includes phosphoric acid or its salt and citric acid or its salt, particularly preferably phosphoric acid and citric acid, for adjusting the pH of the liquid oral composition to 3.0 to 4.0 while suppressing the acidity and enhancing the flavor of the composition.

The salt of citric acid is not limited. Examples include trisodium citrate, tripotassium citrate, and calcium citrate. When citrate is used, only one citrate may be used alone or two or more citrates may be used in combination.

Phosphoric acid includes not only orthophosphoric acid but also condensed phosphoric acids such as pyrophosphoric acid, polyphosphoric acid, metaphosphoric acid, tripolyphosphoric acid, tetrametaphosphoric acid, pentametaphosphoric acid, and hexametaphosphoric acid. In the present invention, one or a combination of these compounds can be used as phosphoric acid. The salt of phosphoric acid is not limited. Examples include sodium pyrophosphate (also known as sodium diphosphate hydrate or anhydride including, for example, sodium diphosphate decahydrate and tetrasodium pyrophosphate (anhydrous)), sodium acid pyrophosphate (also known as disodium dihydrogen pyrophosphate), potassium pyrophosphate (also known as tetrapotassium pyrophosphate), sodium tripolyphosphate (also known as sodium triphosphate), sodium polyphosphate, potassium polyphosphate, sodium trimetaphosphate, sodium tetrametaphosphate, sodium pentametaphosphate, sodium hexametaphosphate (also known as sodium metaphosphate (in Japan's Specifications and Standards for Food Additives and the like)), sodium acid metaphosphate (also known as sodium hydrogen metaphosphate), sodium acid hexametaphosphate, sodium ultraphosphate, potassium metaphosphate, and mixtures of any of these.

Phosphoric acid or its salt is preferably orthophosphoric acid or its salt, more preferably orthophosphoric acid.

The amount of the acidulant can be set according to, for example, the type of the acidulant. The acidulant may be used in an amount that allows the liquid oral composition to have a pH of 3.0 to 4.0. For example, the amount of the acidulant in the liquid oral composition is preferably 100 to 3000 mg/100 mL, more preferably 300 to 2000 mg/100 mL, as the total amount of the acidulant in terms of the free acid content. The amount means the total amount when several types of acidulants are used. In regard to the expression “the amount in terms of the free acid content” or its similar expression used herein, when an acid is in the free acid form, such an expression means the free acid content, and when the acid is in the salt form, such an expressions means the value obtained by multiplying the number of moles of the salt by the molecular weight of the corresponding free acid.

In one embodiment, when citric acid or its salt and phosphoric acid or its salt are used as acidulants, the amount of citric acid or its salt in the liquid oral composition is preferably 100 to 2000 mg/100 mL, more preferably 200 to 1500 mg/100 mL, still more preferably 300 to 1500 mg/100 mL, as the total amount of citric acid or its salt in terms of free acid content, in order to moderate the acidity and in terms of the flavor of the liquid oral composition. The amount of phosphoric acid or its salt in the liquid oral composition is preferably 100 to 1000 mg/100 mL, more preferably 120 to 900 mg/ mL, still more preferably 200 to 800 mg/100 mL, as the total amount of phosphoric acid or its salt in terms of free acid content.

In order to moderate the acidity and in terms of the flavor of the liquid oral composition, the weight ratio of the total weight of phosphoric acid or its salt in terms of free acid content to the total weight of citric acid or its salt in terms of free acid content (total weight of phosphoric acid or its salt in terms of free acid content:total weight of citric acid or its salt in terms of free acid content) is preferably 1:1 to 1:5, more preferably 1:1 to 1:3.

In another embodiment, when the acidulant is citric acid or its salt, the amount of citric acid or its salt in the liquid oral composition is preferably 100 to 2000 mg/100 mL, more preferably 200 to 1500 mg/100 mL, as the total weight of citric acid or its salt in terms of free acid content.

### <Other Components and the Like>

The liquid oral composition of the present invention may contain one or more components other than those described above as long as the effect of the present invention is not impaired.

Preferably, the liquid oral composition of the present invention contains a sweetener. Adding a sweetener can add adequate sweetness, further moderating the acidity of the liquid oral composition and providing a better flavor. Any sweetener may be used. Examples include sugars, sugar alcohols, and high-intensity sweeteners. These can be used alone or in combination of two or more thereof.

Examples of the sugars include monosaccharides, disaccharides, trisaccharides, and higher polysaccharides (including oligosaccharide). Specific examples include glucose, fluctose, galactose, mannose, sucrose, maltose, lactose, and trehalose. Examples of the sugar alcohols include erythritol, xylitol, sorbitol, mannitol, maltitol, and reduced palatinose. Of these, erythritol is more preferred. The amount (total amount) of sugar and sugar alcohol in the liquid oral composition is preferably 1000 to 15000 mg/100 mL, more preferably 3000 to 10000 mg/100 mL, for adding adequate sweetness and providing a better flavor.

The high-intensity sweeteners mean sweeteners having higher sweetness than sugars. Specific examples include acesulfame potassium (acesulfame K), sucralose, aspartame, stevia, saccharin, sodium saccharin, and neotame. Of these, acesulfame K and sucralose are preferred. The amount of high-intensity sweetener in the liquid oral composition is preferably 1 to 50 mg/100 mL, more preferably 3 to 30 mg/100 mL, for adding adequate sweetness and providing a better flavor. The amount means the total amount when several types of high-intensity sweeteners are used.

In one embodiment, the liquid oral composition of the present invention preferably contains a sugar or a sugar alcohol and a high-intensity sweetener, and more preferably contains acesulfame K, sucralose, and erythritol. Adding these sweeteners enhances the flavor of the liquid oral composition. In one embodiment, in view of flavor, the liquid oral composition preferably contains acesulfame K, sucralose, and erythritol in the above amount.

In addition to the collagen peptides, the liquid oral composition of the present invention may also contain other biofunctional materials such as a material known to have a skin improving effect. Examples of the material known to have a skin improving effect include proteoglycans, elastin peptides, ceramides, plant extracts, chondroitin sulfates, glucosamines, minerals (e.g., calcium), and vitamins (e.g., L-ascorbic acid (vitamin C)).

In one embodiment, the liquid oral composition may contain a proteoglycan and/or elastin peptide. Adding a proteoglycan and/or elastin peptide may result in a stronger acidic taste when the liquid oral composition has a pH of 3.0 to 4.0. The present invention can moderate the acidity of the liquid oral composition containing collagen peptides in the pH range of pH 3.0 to 4.0 even when the liquid oral composition further contains a proteoglycan and/or elastin peptide.

The proteoglycan is a general term for compounds in which glycosaminoglycans (mucopolysaccharides) such as chondroitin sulfate and dermatan sulfate are covalently bonded to the core protein. Proteoglycans are found in connective tissues in cartilages and skins of animals, and are essential substances to maintain the structure of these tissues.

The type, source, and production method of the proteoglycan used in the present invention are not limited. For example, proteoglycans extracted from cartilage of fish such as shark, salmon, or ray can be used. Of these, proteoglycans extracted from salmon, particularly from nasal cartilage of salmon, is preferred. Such a proteoglycan can be used alone or in combination with proteoglycans from other sources. Commercially available proteoglycans may be used.

The amount of the proteoglycan in the liquid oral composition of the present invention is preferably 1 to 200 mg/100 mL, more preferably 5 to 100 mg/100 mL. The amount means the total amount when several types of proteoglycans are used.

In the present invention, the elastin peptide means water-soluble elastin peptide. Examples of the elastin peptide include those extracted from biological tissues of animals such as bovine, swine, chicken, sheep, and fish, and decomposition products obtained by, for example, hydrolyzing water-soluble or water-insoluble elastin with an enzyme, acid, alkali, or the like. Artificially synthesized elastin peptide may also be used. One type thereof may be used alone or two or more types thereof may be used.

The molecular weight of the elastin peptide used in the present invention is not limited. Elastin peptides of any molecular weight can be used.

Commercially available elastin peptide may be used. Examples include “Bonito Elastin” (Hayashikane Sangyo Co., Ltd.), “BIDAN Elastin FI” (Nippon Suisan Kaisha, Ltd.), “Tuna Elastin HS-1” (Hagoromo Foods Corporation), and “P-Elastin” (NH Foods Ltd.).

In the liquid oral composition of the present invention, the amount of the elastin peptide is preferably 10 to 750 mg/100 mL, more preferably 50 to 300 mg/100 mL. The amount means the total amount when several types of elastin peptide are used.

In addition to those mentioned above, the liquid oral composition of the present invention may also contain additives such as antioxidants, stabilizers, preservatives, flavorings, emulsifiers, pigments, seasonings, pH adjusters, and nutritional enhancer as long as the effect of the present invention is not impaired.

The liquid oral composition of the present invention contains an aqueous medium (usually water). Preferably, the liquid oral composition of the present invention is a liquid oral composition (aqueous liquid oral composition) that uses water as a medium.

The term “liquid” in the liquid oral composition of the present invention means the liquid state at room temperature. The liquid composition is preferably a fluid having a viscosity (22° C.) of about 500 mPa·s or less. In the present invention, the viscosity of the liquid oral composition is the viscosity at 22° C., and can be measured using a B-type viscometer by a method described in Examples.

Preferably, the liquid oral composition of the present invention has a viscosity of 30 to 500 mPa·s. When the viscosity of the liquid oral composition is in this range, the acidity of the composition can be further moderated in the pH range of 3.0 to 4.0. In addition, when the viscosity of the liquid oral composition is in the above range, the liquid oral composition has a viscosity suitable for drinking when provided as a beverage, for example. Thus, the viscosity in the above range is preferred. The viscosity of the liquid oral composition is preferably 30 mPa·s or more, more preferably 40 mPa·s or more, still more preferably 50 mPa·s or more, while it is preferably 500 mPa·s or less, more preferably 250 mPa·s or less, still more preferably 150 mPa·s or less. In one embodiment, in order to moderate the acidity and achieve a viscosity suitable for drinking, the viscosity of the liquid oral composition is more preferably 40 to 250 mPa·s, still more preferably 50 to 150 mPa·s. In one embodiment of the present invention, adding welan gum in an amount that allows the liquid oral composition to have a viscosity in the above range is preferred.

The pH herein is the pH at 25° C. The pH of the liquid oral composition is preferably 3.9 or lower, more preferably 3.8 or lower while it is preferably 3.2 or higher, more preferably 3.4 or higher, for allowing the acidity moderating effect to be more sufficiently exerted. In one embodiment, the pH of the liquid oral composition is preferably 3.2 to 3.9, more preferably 3.4 to 3.9, still more preferably 3.4 to 3.8.

The method of producing the liquid oral composition of the present invention is not limited. For example, the method preferably includes a mixing step of mixing the components and a pH adjusting step of adjusting the pH of the composition to 3.0 to 4.0.

In the mixing step, preferably, the components are mixed by adding an aqueous medium. The aqueous medium is usually water. The order of mixing the components is not limited as long as the components are mixed uniformly. In one embodiment, when a volatile component (e.g., flavoring) or an easily degradable component (e.g., vitamin C) is added, preferably, such a component is added last. The pH adjusting step can be performed by adding an acidulant to the composition. The pH adjusting step may be performed simultaneously with or after the mixing step. The method of producing the liquid oral composition may include a step such as a viscosity adjusting step of adjusting the viscosity. The viscosity can be adjusted by adding welan gum to the composition. The viscosity adjusting step may be performed simultaneously with the mixing step or the pH adjusting step. When adding a powdered raw material to the composition containing welan gum, preferably, a solution of the powdered raw material is added to the composition, for facilitating the work to uniformly dissolve the raw material in the composition.

The liquid oral composition of the present invention is preferably used as a beverage (beverage composition).

The liquid oral composition of the present invention can be packed in a container. The form of the container is not limited. A sealed container such as a bottle, can, plastic bottle, paper pack, aluminum pouch, or plastic pouch can be used to pack the liquid oral composition to provide a beverage in a container (packaged beverage) or the like.

### <Method of Moderating Acidity>

The present invention also encompasses a method of moderating the acidity of a liquid oral composition, the method including adding welan gum to a liquid oral composition that contains collagen peptides and an acidulant and has a pH of 3.0 to 4.0.

The collagen peptides, acidulant, welan gum, their preferred embodiments, amounts, and the like are the same as those in the liquid oral composition described above. The liquid oral composition may contain other components such as the sweeteners described above.

The method and timing of adding welan gum are not limited as long as the liquid oral composition having a pH of 3.0 to 4.0 ultimately contains the welan gum. In one example, after welan gum is added to a liquid composition containing collagen peptides, the composition may be mixed with an acidulant to adjust the pH to 3.0 to 4.0. In another example, a liquid oral composition containing collagen peptides and an acidulant and having a pH of 3.0 to 4.0 may be mixed with welan gum. A preferred viscosity of the liquid oral composition containing welan gum is the same as the viscosity of the liquid oral composition described above. In one embodiment of the present invention, adding welan gum in an amount that allows the liquid oral composition to have a viscosity in the above range is preferred. Adding welan gum can moderate the acidity of the liquid oral composition containing collagen peptides and an acidulant and having a pH of 3.0 to 4.0.

### EXAMPLES

The following shows examples that specifically describe the present invention, but the present invention is not limited to these examples.

Raw materials used in the examples and comparative examples are as follows.
Collagen peptides: average molecular weight of 500, extracted from fish
Welan gum: VISTOP® W available from San-Ei Gen F.F.I., Inc. Proteoglycans: Proteoglycan F (product name) available from Ichimaru Pharcos Co. Ltd.
Elastin peptide: Bonito Elastin (product name) available from Hayashikane Sangyo Co., Ltd.
Pectin: Genu Pectin JM-150-J (product name) available from Sansyo Co. Ltd.
Xanthan gum: San Ace E-S (product name) available from San-Ei Gen F.F.I., Inc.

The above raw materials were used unless otherwise stated. The phosphoric acid used below is orthophosphoric acid.

The average molecular weight of the collagen peptides was measured by a relative molecular mass measurement method in Chinese National Standards (GB standards) GB/T 22729-2008: oligopeptides powder of marine fish. Reagents for M, 451 and M, 189 were glycine-glycine-tyrosine-arginine (M, 451) and glycine-glycine-glycine (M, 189), respectively.

The total amount of Pro-Hyp (PO) and Hyp-Gly (OG) in the above collagen peptides was about 1.4 wt %.

### Examples 1 to 5

Beverages (liquid oral compositions) of Examples 1 to 5 were produced according to the formulation shown in Table 1. Water was used as a medium. Specifically, the raw materials other than acidulants (citric acid and phosphoric acid) were added to and dissolved in water. The pH of the solution was adjusted to 3.5 by the acidulants, and water was added to give a total of 1000 mL. Table 1 shows the final amount of the acidulants required to adjust the pH and the like. The beverages of Example 1 to 5 each had a weight ratio of phosphoric acid to citric acid of 1:2.5. The obtained solutions (50 mL, each) were dispensed into brown bottles. The bottles were sealed and sterilized by immersion. Thus, the beverages of Examples 1 to 5 were obtained. The amount of each component in Table 1 and Tables 3 to 5 (shown later) is the amount (mg/1000 mL) in 1000 mL of the beverage.

### Comparative Examples 1 to 3

Beverages (liquid oral compositions) were produced by the same method as that of Example 1, except that the raw materials were used in the amounts shown in Table 1. The beverages of Comparative Examples 2 and 3 respectively contained pectin and xanthan gum used as thickeners.

The beverages obtained in Examples 1 to 5 and Comparative Examples 1 to 3 were subjected to sensory evaluation of the flavor at room temperature by a method described below. The viscosity and pH of each beverage were measured by methods described below. Table 1 shows the formulation and evaluation results of each beverage. The “Collagen peptides/welan gum” in the table is the weight ratio of the collagen peptides to the welan gum.

### <Flavor Evaluation>

The flavor of the beverage was sensorily evaluated in terms of acidity. Five expert panelists evaluated the acidic mouthfeel and the acidic aftertaste of each beverage based on the criteria shown in Table 2. The acidic mouthfeel is the acidity felt in the mouth when holding a beverage in the mouth (acidity of the initial taste), and the acidic aftertaste is the acidity remaining in the month after drinking a beverage (acidity of the aftertaste or persisting taste).

The results were given based on the average score of the evaluation as follows: 4.0 to 3.1: good; 3.0 to 2.1:

fair; less than 2.1: poor.

### <Viscosity Measurement>

The viscosity of each sample (beverage) was measured using a B-type viscometer by a method described below.

Measurement device (B-type viscometer): BII-type viscometer (Toki Sangyo Co., Ltd.), model BMII
Rotor: No. 2
Sample container: 100-mL sample bottle
Amount of sample: 75 mL

Each sample was maintained at 22° C. The value was read one minute after the start of rotation, and regarded as the viscosity. The rotational speed during the measurement was 60 rpm.

### <pH Measurement>

The pH (25° C.) was measured using a pH meter (model: F-53, available from HORIBA, Ltd.).

TABLE 1

| | | | | | | Comparative | Comparative | Comparative |
|---|---|---|---|---|---|---|---|---|
| | Example 1 | Example 2 | Example 3 | Example 4 | Example 5 | Example 1 | Example 2 | Example 3 |
| Collagen peptides | 50000 | 50000 | 50000 | 50000 | 50000 | 50000 | 50000 | 50000 |
| Welan gum | 750 | 1125 | 1500 | 2000 | 2500 | — | — | — |
| Acidulant (citric acid) | 6190 | 6190 | 6190 | 6190 | 6190 | 6190 | 6190 | 6190 |
| Acidulant (phosphoric acid) | 2470 | 2470 | 2470 | 2470 | 2470 | 2470 | 2470 | 2470 |
| L-ascorbic acid | 3600 | 3600 | 3600 | 3600 | 3600 | 3600 | 3600 | 3600 |
| Erythritol | 60000 | 60000 | 60000 | 60000 | 60000 | 60000 | 60000 | 60000 |
| Sucralose | 96 | 96 | 96 | 96 | 96 | 96 | 96 | 96 |
| Acesulfame K | 120 | 120 | 120 | 120 | 120 | 120 | 120 | 120 |
| Pectin | — | — | — | — | — | — | 3000 | — |
| Xanthan gum | — | — | — | — | — | — | — | 1000 |
| | mg/1000 mL | | | | | | | |
| Collagen peptides/welan gum | 66.7 | 44.4 | 33.3 | 25.0 | 20.0 | — | — | — |
| pH | 3.5 | 3.5 | 3.5 | 3.5 | 3.5 | 3.5 | 3.5 | 3.5 |
| Viscosity (mPa · s) | 37.5 | 68.0 | 110.5 | 174 | 247 | 5.0 | 6.5 | 37.0 |
| Acidic mouthfeel | Fair | Good | Good | Good | Good | Poor | Poor | Fair |
| Acidic aftertaste | Fair | Good | Good | Fair | Fair | Poor | Poor | Poor |

TABLE 2
Criteria for acidity

| Score | Acidic mouthfeel | Acidic aftertaste |
|---|---|---|
| 4 | Hardly any acidity | Good crispiness |
| 3 | Not much acidity | Rather good crispiness |
| 2 | Some acidity | Rather poor crispiness |
| 1 | Very strong acidity | Poor crispiness |

The beverage of Comparative Example 1 had a strong acidic mouthfeel and a strong acidic aftertaste. The beverage of Comparative Example 2 to which pectin was added had the same results. The beverage of Comparative Example 3 to which xanthan gum was added had a less acidic mouthfeel but had a strong acidic aftertaste. The beverages of Examples 1 to 5 to which welan gum was added each had a less acidic mouthfeel and a less acidic aftertaste. The beverages of Examples 1 to 5 each had an easily drinkable viscosity.

### Examples 6 and 7

Beverages of Examples 6 and 7 were produced according to the formulation shown in Table 3 by the same method as that of Example 1 in order to evaluate the acidity moderating effect produced by addition of a proteoglycan or elastin peptide in addition to the collagen peptides.

The beverages of Example 6 and 7 each had a weight ratio of phosphoric acid to citric acid of 1:2.5.

The flavor of each of the beverages obtained in Examples 6 and 7 was evaluated at room temperature as in Example 1. The viscosity and pH of the beverages were measured by the above methods. Table 3 shows the evaluation results.

The beverages of Examples 6 and 7 each also had a less acidic mouthfeel and a less acidic aftertaste.

TABLE 3

| | Example 6 | Example 7 |
|---|---|---|
| Collagen peptides | 50000 | 50000 |
| Welan gum | 1500 | 1500 |
| Proteoglycans | 500 | — |
| Elastin peptides | — | 1500 |
| Acidulant (citric acid) | 6190 | 6190 |
| Acidulant (phosphoric acid) | 2470 | 2470 |
| L-ascorbic acid | 3600 | 3600 |
| Erythritol | 60000 | 60000 |
| Sucralose | 96 | 96 |
| Acesulfame K | 120 | 120 |

mg/1000 mL

| | | |
|---|---|---|
| Collagen peptides/welan gum | 33.3 | 33.3 |

| | |
|---|---|
| pH | 3.5 |

| | | |
|---|---|---|
| Viscosity (mPa · s) | 119 | 112 |
| Acidic mouthfeel | Fair | Good |
| Acidic aftertaste | Good | Fair |

### Examples 8 to 10

Beverages of Examples 8 to 10 were produced by the same method as that of Example 1, except that the raw materials were used in the amounts shown in Table 4. The beverages of Examples 8 to 10 each had a weight ratio of phosphoric acid to citric acid of 1:2.5.

The flavor of each of the beverages obtained in Examples 8 to 10 was evaluated at room temperature by the same method as that of Example 1. The viscosity and pH of the beverages were measured by the above methods. Table 4 shows the evaluation results.

For comparison, a beverage having a pH of 4.0 was produced with the same formulation as in Example 10, except that welan gum was not added, and the flavor was evaluated in the same manner. The beverage not containing welan gum (pH 4.0) had an acidic mouthfeel and an acidic aftertaste. The beverages of Examples 8 to 10 each had a less acidic mouthfeel and a less acidic aftertaste.

TABLE 4

| | Example 8 | Example 9 | Example 10 |
|---|---|---|---|
| Collagen peptides | 50000 | 50000 | 50000 |
| Welan gum | 2000 | 1500 | 1500 |
| Acidulant (citric acid) | 12200 | 9590 | 3180 |
| Acidulant (phosphoric acid) | 4860 | 3820 | 1270 |
| L-ascorbic acid | 3600 | 3600 | 3600 |
| Erythritol | 60000 | 60000 | 60000 |
| Sucralose | 96 | 96 | 96 |
| Acesulfame K | 120 | 120 | 120 |
| | mg/1000 mL | | |
| Collagen peptides/welan gum | 25.0 | 33.3 | 33.3 |
| pH | 3.0 | 3.3 | 4.0 |
| Viscosity (mPa · s) | 167 | 110.5 | 114 |
| Acidic mouthfeel | Fair | Fair | Good |
| Acidic aftertaste | Fair | Fair | Good |

### Example 11

A beverage was produced by the same method as that of Example 1, except that the raw materials were used in the amounts shown in Table 5 and the pH was adjusted with citric acid.

### Comparative Example 4

A beverage was produced by the same method as that of Example 1, except that the raw materials were used in the amounts shown in Table 5.

The flavor of each of the beverages obtained in Example 11 and Comparative Example 4 was evaluated at room temperature by the same method as that of Example 1. The viscosity and pH of the beverages were measured by the above methods. Table 5 shows the evaluation results.

The beverage of Comparative Example 4 had strong citric acidity, a strong acidic mouthfeel, and a strong acidic aftertaste. The beverage of Example 11 had a less acidic mouthfeel and a less acidic aftertaste than the beverage of Comparative Example 4. Adding welan gum moderated the acidity in the pH range of 3.0 to 4.0, even when only citric acid having a strong acidity was used as the acidulant.

TABLE 5

| | Example 11 | Comparative Example 4 |
|---|---|---|
| Collagen peptides | 50000 | 50000 |
| Welan gum | 1500 | — |
| Acidulant (citric acid) | 8600 | 8600 |
| L-ascorbic acid | 3600 | 3600 |
| Erythritol | 60000 | 60000 |
| Sucralose | 96 | 96 |
| Acesulfame K | 120 | 120 |
| | mg/1000 mL | |
| Collagen peptides/welan gum | 33.3 | — |
| pH | 3.5 | 3.5 |
| Viscosity (mPa · s) | 109 | 4.5 |
| Acidic mouthfeel | Fair | Poor |
| Acidic aftertaste | Fair | Poor |

### Example 12

Collagen peptides having an average molecular weight of 931 were used. A beverage of Example 12 was produced according to the formulation shown in Table 6 by the same method as that of Example 1. The amount of each component in Table 6 is the amount (mg/1000 mL) in 1000 mL of the beverage. The viscosity and pH of the beverages were measured by the above methods. The flavor of the obtained beverage was evaluated at room temperature by the same method as that of Example 1. Table 6 shows the evaluation results.

### Comparative Example 5

The collagen peptides having an average molecular weight of 931 used in Example 12 were used. A beverage was produced by the same method as that of Example 12, except that the raw materials were used in the amounts shown in Table 6, and was evaluated in the same manner. Table 6 shows the evaluation results.

TABLE 6

| | Example 12 | Comparative Example 5 |
|---|---|---|
| Collagen peptides | 50000 | 50000 |
| Welan gum | 1500 | — |
| Acidulant (citric acid) | 6190 | 6190 |
| Acidulant (phosphoric acid) | 2470 | 2470 |
| L-ascorbic acid | 3600 | 3600 |
| Erythritol | 60000 | 60000 |
| Sucralose | 96 | 96 |
| Acesulfame K | 120 | 120 |
| | mg/1000 mL | |
| Collagen peptides/welan gum | 33.3 | — |
| pH | 3.5 | 3.5 |
| Viscosity (mPa · s) | 109 | 5 |
| Acidic mouthfeel | Good | Poor |
| Acidic aftertaste | Good | Poor |

The liquid oral composition of Comparative Example 5 had a strong acidic mouthfeel and a strong acidic aftertaste. The acidity was moderated by adding welan gum.

### INDUSTRIAL APPLICABILITY

The present invention is useful in the food and beverage field and the like.

1. A liquid oral composition comprising:
a collagen peptide;
welan gum; and
an acidulant,
the liquid oral composition having a pH of 3.0 to 4.0.

2. The liquid oral composition according to claim 1,
wherein the amount of the collagen peptide is 600 to 20000 mg/100 mL.

3. The liquid oral composition according to claim 1,
wherein the weight ratio of the collagen peptide to the welan gum (collagen peptide/welan gum) is 20 to 100.

4. The liquid oral composition according to claim 1,
wherein the viscosity is 30 to 500 mPa·s.

5. The liquid oral composition according to claim 1,
wherein the amount of the welan gum is 50 to 400 mg/100 mL.

6. The liquid oral composition according to claim 1,
wherein the acidulant comprises citric acid or its salt and/or phosphoric acid or its salt.

7. The liquid oral composition according to claim 1, further comprising a sweetener.

8. The liquid oral composition according to claim 1, further comprising a proteoglycan and/or elastin peptide.

9. The liquid oral composition according to claim 1,
which is a beverage.

10. A method of moderating the acidity of a liquid oral composition, the method comprising:
adding welan gum to a liquid oral composition that contains a collagen peptide and an acidulant and has a pH of 3.0 to 4.0.
